(19) United States
(12) Patent Application Publication
(10) Pub. No.: US 2024/0390341 A1
(43) Pub. Date: Nov. 28, 2024

(54) METHODS FOR TREATING ADRENAL GLAND TUMORS

(71) Applicant: Corcept Therapeutics Incorporated, Menlo Park, CA (US)

(72) Inventor: Andreas Moraitis, Sunny Isles Beach, FL (US)

(73) Assignee: Corcept Therapeutics Incorporated, Menlo Park, CA (US)

(21) Appl. No.: 18/670,160

(22) Filed: May 21, 2024

Related U.S. Application Data
(60) Provisional application No. 63/468,082, filed on May 22, 2023.

(51) Int. Cl. A61K 31/437; A61K 9/00; A61P 35/00

(52) U.S. Cl. CPC A61K 31/437; A61K 9/0053; A61P 35/00

(57) ABSTRACT

Methods and uses for treating an adrenal gland tumor in a subject suffering therefrom are disclosed, to reduce the size or tumor load of, prevent the growth of, or slow the growth of, or reduce or alter secretions from, or induce degeneration or apoptosis in, the adrenal gland tumor. The novel treatments include use of, or administration of, an effective amount of the glucocorticoid receptor modulator (GRM) compound having a heteroaryl-ketone fused azadecalin structure to a patient suffering from an adrenal gland tumor. The adrenal gland tumor may be a benign adrenal gland tumor. The GRM may be relacorilant, which is ((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone).

The GRM may be orally administered. The GRM may be administered with food. The treatments may be used in the absence of surgery, or prior to, or during, or after surgery for the adrenal gland tumor. The treatments may be used without adversely affecting normal adrenal tissue.

### CROSS-REFERENCE TO RELATED APPLICATION

This application claims priority to, and the benefit of, U.S. Provisional Application No. 63/468,082 filed May 22, 2023, which application is hereby incorporated by reference herein in its entirety.

### BACKGROUND

The adrenal glands are hormone-secreting glands located on the kidneys. Hormones secreted by the adrenal glands include adrenaline, aldosterone, and cortisol. Cortisol is produced and secreted by the adrenal glands in response to adrenocorticotrophic hormone (ACTH) which is secreted by the pituitary gland. Cortisol levels vary during the course of the day and night, and may be measured in blood (e.g., serum, plasm, or whole blood) and may be measured in the morning (when cortisol levels are typically the highest). Cortisol levels may also be measured in urine (e.g., 24-hour urinary cortisol measurement, which may provide a cortisol measurement less affected by the time of day at which sampling was performed), saliva (e.g., late-night salivary cortisol, when the cortisol levels are typically lowest), and other bodily fluids (e.g., tears and sweat). Cortisol may also be measured after a dexamethasone suppression test, in which cortisol provides a measure of the response of the hypothalamic-pituitary-adrenal axis to externally administered glucocorticoids such as dexamethasone.

Tumors of the adrenal glands often result in altered levels of hormone secretion; excess adrenal hormone secretion can lead to hypertension, blood salt ion complications, blood glucose irregularities, and other serious, and possibly life-threatening, disorders. For example, excess cortisol secretion due to an adrenal tumor may lead to excess cortisol, a cause of Cushing's syndrome and other disorders. Cushing's syndrome is characterized by, among other symptoms, hypercortisolism, hypertension, hypercoagulopathy, hyperglycemia, glucose intolerance, obesity, dyslipidemia, muscle weakness, cognitive dysfunction, osteoporosis (e.g., low bone density with increased risk of fractures), skin lesions, upper body fat, moon face, depression, and other symptoms. In addition, cortisol has immunosuppressive effects.

Excess cortisol, and symptoms of Cushing's syndrome, may also be caused by tumors of the pituitary (Cushing's syndrome due to a pituitary tumor is termed “Cushing's Disease” (CD)). However, pituitary control of cortisol production is indirect, being due to secretion of ACTH by the pituitary gland, in contrast to the direct secretion of cortisol by many adrenal gland tumors. Thus, unlike cortisol secretion due to pituitary tumors, cortisol secretion by adrenal gland tumors is typically independent of ACTH levels.

Benign adrenal gland tumors typically increase in size over time. Malignant adrenal gland tumors increase in size over time, and may metastasize as well. Thus, treatments that slow or prevent the growth of adrenal gland tumors, or which reduce the size of adrenal gland tumors, are needed.

Conventional treatments for adrenal gland tumors typically include surgery (which may be minimally-invasive laparoscopic surgery). However, since the adrenal glands are critical to health, surgical excision of adrenal gland tumors may lead to loss of adrenal function and deleterious side-effects.

Accordingly, there is need in the art to provide improved treatments for adrenal gland tumors, including adrenal gland tumors that are responsible for cortisol excess in patients suffering from such tumors.

### SUMMARY

Methods and uses are disclosed herein for reducing the adrenal gland tumor load in a patient suffering from an adrenal gland tumor. Such reduction in tumor load may be accomplished without adversely affecting normal adrenal tissue. Reducing adrenal gland tumor load includes, in embodiments, reducing the size of, reducing the volume of, preventing the growth of, slowing the growth of, causing degenerative changes in, inducing apoptosis in at least part of, reducing secretion from, altering the composition of hormones secreted from, and reducing adverse symptoms caused by, an adrenal gland tumor in a patient suffering from an adrenal gland tumor. The adrenal gland tumor may be a benign adrenal gland tumor, and may be a hormone-secreting adrenal gland tumor, e.g., may be a cortisol-secreting adrenal gland tumor, or an ACTH-secreting adrenal gland tumor, and may secrete both cortisol and ACTH. The methods and uses disclosed herein are believed to provide improved treatments for patients suffering from adrenal gland tumors by reducing the size of, or reducing or preventing growth of, those tumors, and to provide improved treatments for adrenal gland tumors that secrete hormones by reducing the amounts of, or altering the type or types of, or relative amounts of, hormones secreted by the tumors due to tumor shrinkage or reduced or prevented growth due to the treatment or to the use.

The methods disclosed herein comprise the administration of a glucocorticoid receptor modulator (GRM) comprising a heteroaryl-ketone fused azadecalin structure to a patient suffering from an adrenal gland tumor. The uses disclosed herein comprise the use of a GRM comprising a heteroaryl-ketone fused azadecalin structure to reduce the size of, or reduce or prevent the growth of, or reduce adverse symptoms caused by, an adrenal gland tumor in a patient suffering from an adrenal gland tumor. In embodiments, the GRM comprising a heteroaryl ketone fused azadecalin structure is a compound described and disclosed in U.S. Pat. No. 8,859,774, the entire contents of which is hereby incorporated by reference in its entirety.

In preferred embodiments of the methods and uses, the GRM is relacorilant, which is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone, which has the structure:

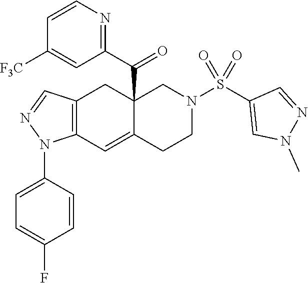 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

In embodiments of the methods disclosed herein, the GRM comprising a heteroaryl-ketone fused azadecalin structure may be orally administered, or may be administered with food, or both. In embodiments of the uses disclosed herein, the use may comprise use of a composition or pharmaceutical formulation for oral delivery of the GRM comprising a heteroaryl-ketone fused azadecalin structure; in embodiments, such a use may comprise use of the composition or pharmaceutical formulation with food.

In embodiments, the adrenal gland tumor is a hormone-secreting tumor (e.g., the adrenal gland tumor may secrete cortisol, or adrenocorticotrophic hormone (ACTH), or other hormone). In embodiments, the adrenal gland tumor is a benign adrenal gland tumor. In embodiments, the patient is treated by administration of the GRM comprising a heteroaryl-ketone fused azadecalin structure in the absence of any other treatment directed at the adrenal gland tumor. Thus, the methods and uses disclosed herein are effective to reduce the size of, slow the growth of, or prevent the growth of an adrenal gland tumor without need for use of, or treatment by, a further anti-cancer treatment.

The methods and uses disclosed herein provide improved treatments for patients suffering from adrenal gland tumors by reducing the size of, or preventing or slowing the growth of, those tumors. The methods and uses disclosed herein are believed to provide improved treatments for patients suffering from hormone-secreting adrenal gland tumors who have symptoms of Cushing's syndrome by reducing those Cushing's syndrome symptoms due to tumor shrinkage due to the treatment or to the use.

### BRIEF DESCRIPTION OF THE DRAWINGS

FIG. 1 shows the study design of GRADIENT, a randomized, double-blind, placebo-controlled Phase 3 study to evaluate the efficacy and safety of relacorilant ((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone). The GRADIENT study focuses on patients with hypercortisolism secondary to adrenal adenoma(s) or hyperplasia. Patients discussed herein were participants in the GRADIENT study.

FIG. 2 presents adrenal scans obtained from a patient with a benign adrenal adenoma (images are computed tomography (CT) images of the patient's abdomen, including the adrenal glands). The image on the left was taken prior to initiation of relacorilant administration. Relacorilant administration began with 100 mg/day dosing, slowly titrated up to 400 mg/day in 100 mg increments. The image on the right was taken after the patient had received daily relacorilant administration for almost two years. The adenoma has decreased in size as compared to the initial size of 4.2 centimeters (cm) to 3.1 cm, and now exhibits areas of degeneration (fat droplets, seen as small black areas inside the adenoma) that were not present in the initial scan. Arrows point to the adrenal tumor in both images. The arrow in the right image it also points a degenerative area inside the tumor that suggests an apoptotic process.

FIG. 3 presents adrenal scans obtained from another patient with a benign adrenal adenoma (images are computed tomography (CT) images of the patient's abdomen, including the adrenal glands). The arrows point the tumor, and the measurement lines (offset from the images for clarity) indicate the maximum dimension of the tumor. The upper image was taken prior to treatment with relacorilant. The measurement line indicates 34.09 millimeters (mm) indicates the largest diameter of the tumor prior to treatment. The lower image was taken after the patient had received at least 3 months of daily relacorilant, beginning at a daily relacorilant doses of 100 mg/day, increased in 100 mg/day steps up to 400 mg/day. The bar in the lower image indicates a reduced tumor dimension of 30.99 mm following treatment with relacorilant.

### DETAILED DESCRIPTION

Methods and uses of a glucocorticoid receptor modulator (GRM) comprising a heteroaryl-ketone fused azadecalin structure for the treatment of a patient suffering from an adrenal gland tumor are disclosed herein. In embodiments, the methods include administering an effective amount of a GRM comprising a heteroaryl-ketone fused azadecalin structure to a patient suffering from an adrenal gland tumor, to reduce the size, or volume, or tumor load of the adrenal gland tumor. Such treatment of the adrenal gland tumor treats the tumor without adversely affecting normal adrenal tissue. In embodiments, the uses include the use of an effective amount of a GRM comprising a heteroaryl-ketone fused azadecalin structure to reduce the size, or volume, or tumor load of an adrenal gland tumor in a patient suffering from that adrenal gland tumor. Such uses treat the tumor without adversely affecting normal adrenal tissue. Thus, in embodiments of the methods and uses, treatment of an adrenal gland tumor comprises treatment to reduce the size of the adrenal gland tumor, treatment to reduce the volume of the adrenal gland tumor, or to reduce the adrenal gland tumor load in a patient suffering from an adrenal gland tumor, or to prevent the growth of that tumor, or to slow the growth of that tumor, or causing degenerative changes in, or inducing apoptosis in at least part of, or reducing secretion from, or to reduce or alter hormone secretion from that tumor, or altering the composition of hormones secreted from, or otherwise reducing the adverse effects of, an adrenal gland tumor in a patient suffering from an adrenal gland tumor.

The methods and uses disclosed herein may be used without surgery for the adrenal gland tumor; or may be used prior to surgery for the adrenal gland tumor; or may be used during surgery for the adrenal gland tumor; or may be used after surgery for the adrenal gland tumor. The methods and uses disclosed herein do not require, and may be performed or used, in the absence of cancer chemotherapy, or radiation, or other cancer treatment.

Reducing the size of adrenal gland tumor may include reducing a dimension of an adrenal gland tumor (e.g., reducing tumor width, or breadth, or length, or height; reducing a cross-sectional diameter or chord of an image of the tumor, or reducing other dimension of the tumor); reducing the volume of an adrenal gland tumor; or reducing other measure indicative of the size of an adrenal gland tumor.

Reducing the adrenal gland tumor load comprises one or more of reducing the volume of, reducing the mass of, reducing the weight of, one or more adrenal gland tumor(s) in a patient suffering from an adrenal gland tumor. In embodiments, the methods and uses disclosed herein may cause regression of an adrenal gland tumor, including causing regression of a cortisol-secreting adrenal adenoma. In embodiments, the methods and uses disclosed herein may cause degenerative changes in an adrenal gland tumor, including causing degenerative changes in a cortisol-secreting adrenal adenoma. In embodiments, causing degenerative changes in an adrenal gland tumor include causing or enhancing apoptosis in the tumor.

In embodiments, preventing the growth of an adrenal gland tumor may comprise preventing the increase in a dimension of an adrenal gland tumor compared to a previous measure of that dimension (where a dimension may be, e.g., tumor width, or length, or height; a cross-sectional diameter or chord of an image of the tumor; the volume of the tumor; the weight of the tumor; or any other dimension related to the size of the tumor). In embodiments, slowing the growth of an adrenal gland tumor may comprise slowing the rate of increase, in a dimension of an adrenal gland tumor over time as compared to the rate of increase, in that dimension over a prior period of the same amount of time (where a dimension may be, e.g., tumor width, or length, or height; a cross-sectional diameter or chord of an image of the tumor; the volume of the tumor; the weight of the tumor; or any other dimension related to the size of the tumor). In embodiments, preventing the growth of an adrenal gland tumor may comprise preventing the increase the tumor load of adrenal gland tumor(s) in a patient as compared to the prior tumor load; and slowing the growth of an adrenal gland tumor may comprise slowing the increase in the tumor load of adrenal gland tumor(s) in a patient as compared to the increase in tumor load over a prior period of the same amount of time. In embodiments, preventing, or slowing, the growth of an adrenal gland tumor in a patient suffering from an adrenal gland tumor may be determined by other measures of tumor size or growth.

In embodiments, the adrenal gland tumor is a benign adrenal gland tumor. In embodiments, the adrenal gland tumor is a malignant adrenal gland tumor. The adrenal gland tumor may be a hormone-secreting adrenal gland tumor, e.g., may be a cortisol-secreting adrenal gland tumor, or an ACTH-secreting adrenal gland tumor, or may secrete one or more other hormones. In embodiments, the methods and uses disclosed herein are effective to reduce secretion from, or alter the composition of hormones secreted from, or otherwise reduce the adverse effects of, a hormone-secreting adrenal gland tumor. In embodiments, the methods and uses disclosed herein are effective to reduce the secretion of cortisol from a cortisol-secreting adrenal gland tumor. Thus, the methods and uses disclosed herein are believed to be effective to reduce cortisol blood levels in the patient in need thereof, to treat and reduce the severity of cortisol excess, symptoms of Cushing's syndrome, or both cortisol excess and Cushing's syndrome symptoms, in the patient suffering from an adrenal gland tumor. In embodiments, a patient suffering from an adrenal gland tumor may exhibit symptoms of Cushing's syndrome; the methods and uses disclosed herein are believed to be effective to treat and ameliorate (e.g., reduce the severity of) symptoms of Cushing's syndrome in the patient. In embodiments, such symptoms of Cushing's syndrome include hypercortisolism, hypertension, hypercoagulopathy, hyperglycemia, glucose intolerance, obesity, dyslipidemia, muscle weakness, cognitive dysfunction, osteoporosis (e.g., low bone density with increased risk of fractures), skin lesions, upper body fat, moon face, depression, a suppressed immune system, and other symptoms.

Cortisol-secreting adrenal gland tumors typically lead to cortisol excess, including leading to abnormally high levels of cortisol in the blood of the patient, and leading to a lack of appropriate diurnal rhythm in the patient's cortisol levels. Since ACTH is the hormone that signals the adrenal glands to secrete cortisol, ACTH-secreting tumors typically cause the same symptoms as adrenal gland tumors that directly secrete cortisol. In embodiments, the patient suffering from an adrenal gland tumor also suffers from Cushing's syndrome. In embodiments, reducing the size of a hormone-secreting adrenal gland tumor may be effective to reduce the amount of a hormone or of multiple hormones secreted by the tumor; and reducing the size of a hormone-secreting adrenal gland tumor may be effective to alter the composition, or to alter the type(s) of hormone(s) secreted from that tumor. In embodiments, reducing the size of a hormone-secreting adrenal gland tumor may be effective to reduce Cushing's syndrome symptoms in the patient by reducing the size of that tumor. Thus, the methods and uses disclosed herein are believed to provide effective treatment for such cortisol excess. The methods and uses disclosed herein are believed to provide effective treatment for Cushing's syndrome and symptoms of Cushing's syndrome in a patient suffering from a cortisol-secreting or ACTH-secreting adrenal gland tumor, by reducing the size, or by reducing the amounts of hormone-secretion, of that adrenal gland tumor.

Accordingly, in embodiments, an adrenal gland tumor in a patient treated with the methods disclosed herein may be reduced in size after treatment with, or the use of, a GRM comprising a heteroaryl-ketone fused azadecalin structure; the growth of that tumor may be prevented, or may be slowed; degenerative changes, or apoptosis, may be induced in at least a portion of the tumor; the amount of a hormone secreted by the adrenal gland tumors may be reduced after such treatment or use; the type or types of, or relative amounts of, hormones secreted by the adrenal gland tumors may be altered after such treatment or use; Cushing's syndrome symptoms may be reduced in the patients due to tumor shrinkage due to the treatment or to the use; and patients may experience other benefits from the treatments and uses disclosed herein.

In embodiments of the methods and uses, the GRM comprising a heteroaryl ketone fused azadecalin structure is a compound described and disclosed in U.S. Pat. No. 8,859,774, the entire contents of which is hereby incorporated by reference in its entirety. In embodiments of the methods and uses, the GRM is relacorilant, which is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone, which has the structure:

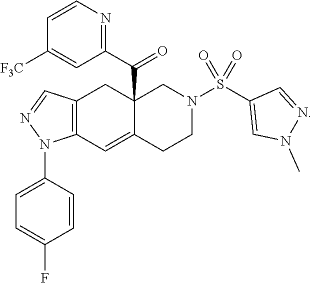 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

In embodiments of the methods and uses, the GRM comprising a heteroaryl ketone fused azadecalin structure is the compound (R)-(1-(4-fluorophenyl)-6-((4-(trifluoromethyl)phenyl) sulfonyl)-4,4a, 5,6,7,8-hexahydro-1-H-pyrazolo P,4-g]isoquinolin-4a-yl)(pyridin-2-yl)methanone, also known as “dazucorilant”, which has the following structure:

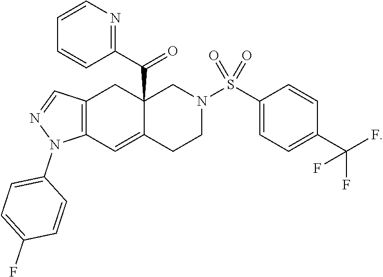 [SMILES: O=C(c1ccccn1)[C@]12Cc3cnn(-c4ccc(F)cc4)c3C=C1CCN(S(=O)(=O)c1ccc(C(F)(F)F)cc1)C2]

The GRM may be orally administered. In embodiments, the GRM may be administered with food.

The effective amount of the GRM comprising a heteroaryl ketone fused azadecalin structure may be a daily dose of between 10 milligrams per day (mg/day) and 1000 mg/day (inclusive; all ranges discussed herein include the limiting values and all values in between the limiting values). In embodiments, the effective amount of a GRM comprising a heteroaryl ketone fused azadecalin structure is a daily dose of between about 15 mg/day and 900 mg/day. In further embodiments, the effective amount is a daily dose of between about 20 mg/day and 800 mg/day. In still further embodiments, the effective amount is a daily dose of between about 25 mg/day and 750 mg/day. In yet further embodiments, the effective amount is a daily dose of between about 30 mg/day and 700 mg/day. In some embodiments, the daily dose of the GRM comprising a heteroaryl ketone fused azadecalin structure is 1, 2, 4, 6, 8, 10, 12, 14, 16, 18, 20, 30, 40, 50 60, 70, 80, 90 or 100 mg/kg/day. In some cases, the GRM comprising a heteroaryl ketone fused azadecalin structure is administrated for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 weeks.

The methods and uses disclosed herein are believed to provide improved treatments for patients suffering from adrenal gland tumors, including hormone-secreting adrenal gland tumors by reducing the size, or tumor load, or slowing or preventing the growth of, or inducing degeneration or apoptosis in, or reducing the amounts of, or altering the type or types of, or relative amounts of, hormones secreted by, the tumors due to tumor shrinkage due to the treatment or to the use. The methods and uses disclosed herein are believed to provide improved treatments for patients suffering from hormone-secreting adrenal gland tumors who have symptoms of Cushing's syndrome by reducing those Cushing's syndrome symptoms due to tumor shrinkage due to the treatment or to the use.

Adrenal gland tumors differ greatly from tumors of the pituitary gland, not only in location and environment, but in structure and function as well. Pituitary macroadenomas have been reported to shrink following administration of relacorilant (see, e.g., U.S. Pat. No. 10,946,005). However, the location, and so biological environment, of a pituitary tumor (in a part of the brain located directly beneath the hypothalamus) differs substantially from the location and environment of an adrenal gland tumor (in the abdominal cavity, located in the adrenal glands, which are attached to, and on top of, the kidneys).

### Definitions

Acronyms used in this application include: ABPM, ambulatory blood pressure monitoring; ACTH, adrenocorticotropic hormone; DBP, diastolic blood pressure; DST, dexamethasone suppression test; DXA, dual-energy X-ray absorptiometry; oGGT, oral glucose tolerance test; SBP, systolic blood pressure; and ULN, upper limit of normal.

The term “about” when used in reference to a pre-determined value denotes a range encompassing plus or minus 10% of the pre-determined value.

As used herein, the term “tumor” refers to an abnormal growth of tissue that results from excessive cell division. A tumor that invades the surrounding tissue and/or can metastasize is referred to as “malignant.” A tumor that does not metastasize is referred to as “benign.”

As used herein, the term “adrenal gland tumor” refers to any tumor, whether benign or malignant, located in or on an adrenal gland.

As used herein, the term “adenoma” refers to a tumor, or hyperplasia, typically benign, and “adrenal adenoma” refers to an adenoma of the adrenal gland (hyperplasia of an adrenal gland). An adenoma may also be termed a “nodule”, and a nodule of the adrenal gland may be termed “an adrenal nodule”.

As used herein, the term “adrenocortical carcinoma” and the acronym “ACC” are used interchangeably to refer to cortical carcinomas of the adrenal gland.

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. Administration may be by oral administration (i.e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramuscular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art. The effective amount can be an amount effective to invoke an antitumor response. For the purpose of this disclosure, the effective amount of SGRM or the effective amount of a chemotherapeutic agent is an amount that would reduce tumor load or bring about other desired beneficial clinical outcomes related to cancer improvement when combined with a chemotherapeutic agent or SGRM, respectively.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “Adrenocorticotrophic Hormone” (ACTH) refers to the peptide hormone produced and secreted by the anterior pituitary gland that stimulates the adrenal cortex to secrete glucocorticoid hormones, which help cells synthesize glucose, catabolize proteins, mobilize free fatty acids and inhibit inflammation in allergic responses. One such glucocorticoid hormone is cortisol, which regulates metabolism of carbohydrate, fat, and protein metabolism. In healthy mammals, ACTH secretion is tightly regulated. ACTH secretion is positively regulated by corticotropin releasing hormone (CRH), which is released by the hypothalamus. ACTH secretion is negatively regulated by cortisol and other glucocorticoids.

The terms “adrenal hormone”, “adrenal pre-hormone”, and “adrenal hormone or adrenal pre-hormone” refer to molecules that are, or are precursors of, hormones produced by the adrenal gland. As used herein, without limitation, an “adrenal hormone or adrenal pre-hormone” may be one or more of aldosterone, other mineralocorticoids, cortisol, 11-deoxycortisol, 11-deoxycorticosterone, corticosterone, 18-hydroxycorticosterone, other endogenous glucocorticoids, epinephrine, other catecholamines, 17α-hydroxy pregnenolone, 17α-hydroxy progesterone, pregnenolone, progesterone, dehydroepiandrosterone (androstenolone, DHEA), dehydroepiandrosterone sulfate (DHEA-S), androstenedione, including their metabolites and precursors.

The term “measuring the level,” in the context of ACTH, cortisol, adrenal hormone, adrenal pre-hormone, or other hormone, refers determining, detecting, or quantitating the amount, level, or concentration of, for example, cortisol, ACTH or other hormone in a sample obtained from a subject. The sample may be, e.g., a blood sample, a saliva sample, a urine sample, or other sample obtained from the patient. A level may be measured from a fraction of a sample. For example, a level (e.g., ACTH or cortisol) may be measured in the plasma fraction of a blood sample; may be measured in a serum fraction of a blood sample; or, in embodiments, may be measured in whole blood.

The term “cortisol” refers to the naturally occurring glucocorticoid hormone (also known as hydrocortisone) that is produced by the zona fasciculata of the adrenal gland. Cortisol has the structure:

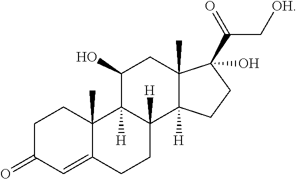

The term “total cortisol” refers to cortisol that is bound to cortisol-binding globulin (CBG or transcortin) and free cortisol (cortisol that is not bound to CBG). The term “free cortisol” refers to cortisol that is not bound to cortisol-binding globulin (CBG or transcortin). As used herein, the term “cortisol” refers to total cortisol, free cortisol, and/or cortisol bound of CBG.

Cortisol levels may be measured in blood (e.g., serum or plasma), urine, saliva, and other bodily fluids. Urinary free cortisol (UFC, a measure of cortisol in urine excreted over 24 hours) is a common cortisol level measurement method, which masks the daily cortisol variations by requiring a full day's sample. Plasma cortisol (a measure of the cortisol levels at the time the blood sample is taken) is often used for dexamethasone suppression testing (which tests patient response to rapid increases in glucocorticoid levels). In addition, cortisol level can also be measured in a serum sample according to methods known in the art. Salivary cortisol may also be measured. The numerical value of the cortisol level differs between measurement methods; that is, blood cortisol levels (e.g., serum or plasma levels) sample cortisol at the time the blood sample is taken, and are numerically different than salivary cortisol levels (sample cortisol at the time the saliva sample is taken) and numerically different than urinary free cortisol levels (which represent cortisol levels over a 24-hour period).

The level of cortisol can be measured in a sample (of, e.g., serum, plasma, saliva, urine, or any other biological fluid) using various methods, including but not limited to, immunoassays, e.g., competitive immunoassay, radioimmunoassay (MA), immunofluorometric enzyme assay, and ELISA; competitive protein-binding assays; liquid chromatography (e.g., HPLC); and mass spectrometry, e.g., high-performance liquid chromatography/triple quadrupole-mass spectrometry (LC-MS/MS). In preferred embodiments, cortisol levels are measured using LC-MS/MS, such as performed by Quest Diagnostics (Secaucus, N.J. 07094).

The term “normal level” refers to the average level of an analyte as determined by measurements of samples obtained from multiple normal subjects. For comparison, the same types of measurements (e.g., plasma or serum; salivary; or urinary) must be the compared.

The term “normal cortisol level” refers to the average level or concentration of cortisol as determined by measurements of samples (e.g., serum samples) obtained from multiple normal subjects (e.g., during random or timed cortisol collection or following a dynamic test (suppression or stimulation testing). For example, as reported by Putignano et al. (European Journal of Endocrinology 145:165-171 (2001)), normal plasma cortisol in healthy women was about 420 nanomoles per liter (nmol/l) at 8 AM (morning); about 250 nmol/l at 5 PM (evening); and about 90 nmol/l at 12 PM (late night). Salivary cortisol measurements from these women were about 14 nmol/l at 8 AM (morning); about 7 nmol/l at 5 PM (evening); and about 5 nmol/l at 12 PM (late night). Urinary free cortisol levels measured in these healthy women were about 130 nmol per 24 hours (nmol/24 h). Cortisol levels are suppressed by the dexamethasone suppression test (DST), as indicated by the plasma cortisol level of about 24 nmol/l following DST and the salivary cortisol level of about 4 nmol/l following DST.

As used herein, the term “cortisol excess” refers to cortisol levels, or cortisol activity, however measured, that are greater than about 1.5 times, or greater than about 2 times, the cortisol levels (or cortisol activity) measured in healthy subjects (where the healthy subject cortisol levels or cortisol activity are measured by the same methods as the patient's cortisol level or cortisol activity is measured). For example, using the morning plasma cortisol levels of Putignano et al., cortisol excess would be determined if a patient had morning plasma cortisol levels of about 630 nmol/l or greater, or of about 840 nmol/l or greater. Using the morning salivary cortisol levels of Putignano et al., cortisol excess would be determined if a patient had morning salivary cortisol levels of about 21 nmol/l or greater, or of about 28 nmol/l or greater. Using the 24-hour urinary free cortisol levels of Putignano et al., cortisol excess would be determined if a patient had 24-hour urinary cortisol levels of about 195 nmol/24 h or greater, or of about 260 nmol/24 h or greater.

There are several variations of the dexamethasone suppression test (DST), including the 1 mg overnight DST, the 8 mg overnight DST, the 2 day low dose DST, and the 2 days high dose DST. Levels of 1.8 microgram per deciliter (mcg/dl) or less were considered normal in evaluating the DST results of the present studies. Applicant notes that lower values (less than 1 mcg/dl DST test results) have also been suggested for use in determining whether or not a subject's DST cortisol levels are normal.

In embodiments, combined criteria may be used to identify patients with cortisol excess. For example, two or more, or all, or the following criteria may be used to determine whether or not a patient suffers from cortisol excess:
1. Urinary Free Cortisol (UFC) greater than the upper limit of normal (ULN)
2. Late night salivary cortisol (LNSC) greater than ULN for 2 nights
3. Dexamethasone Suppression Test (DST) with cortisol level greater than 1 mcg/dL
4. Adrenocorticotrophic Hormone (ACTH) less than 10 picogram per milliliter (pg/mL)

“Standard control” as used herein refers to a sample comprising a predetermined amount of an analyte (such as ACTH or cortisol) suitable for the use of an application of the present invention, in order to serve as a comparison basis for providing an indication of the relative amount of the analyte (e.g., ACTH or cortisol) that is present in a test sample. A sample serving as a standard control provides an average amount of an analyte such as ACTH or cortisol that is representative for a defined sample type (e.g., plasma, serum, saliva, or urine) taken at a defined time of the day (e.g., 8 AM) from an average individual who is not suffering from or at increased risk of later developing hypokalemia or any associated disorder or complication and has been given the same GRM treatment. As used herein, a “blood sample” may be a whole blood sample, serum sample, plasma sample, or blood cell sample as appropriate for measuring an analyte level by art-known methods according to conventional use. Similarly, “blood level” of a particular analyte maybe the level of the analyte in the whole blood, serum, plasma, or blood cells. For example, the blood level of potassium, ACTH, or cortisol maybe the level of each analyte in a serum or plasma sample taken from a subject being tested.

The term “average,” as used in the context of describing an individual (especially a human subject) who does not have and is not at increased risk of developing hypokalemia or any related condition or disorder prior to receiving GRM treatment, refers to certain characteristics, such as the level or amount of an analyte (such as ACTH or cortisol) present in a sample taken from the individual without receiving GRM treatment, that are representative of the average amount or level of the analyte found in a randomly selected group of individual subjects who have not been diagnosed with and are not susceptible to hypokalemia or any related diseases or conditions and therefore can serve as an “average normal value” or “standard control value” for the particular analyte prior to GRM treatment. This selected group should comprise a sufficient number of individuals (e.g., at least 200 or 500 or more) such that the average value (i.e., level or amount) of the analyte of interest (e.g., ACTH or cortisol) assessed among these individuals reflects, with reasonable accuracy, the corresponding level or amount of the analyte found in the general population of non-hypokalemic individuals with no known risk for the disorder or related conditions upon receiving GRM treatment. In some cases, the selected group of individuals generally have the same gender, are similar in age (e.g., within a 5- or 10-year age difference from one another), have similar ethnic and medical backgrounds. Depending on the analyte, the average value or standard control value may need to be ascertained from samples taken from these individuals at about the same time during the day (e.g., 6 AM, 8 AM, 12 PM, 4 PM, or 6 PM). The average or standard control value of any particular analyte may also vary depending on the specific assay or assay format (including the specific reagents) utilized for quantitatively measuring the analyte, and therefore can be made available either by way of experimentation or by way of assay manufacturer's information.

The term “glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

A mineralocorticoid receptor (MR), also known as a type I glucocorticoid receptor (GR I), is activated by aldosterone in humans.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

“Pharmaceutically-acceptable excipient” and “pharmaceutically-acceptable carrier” refer to a substance that aids the administration of an active agent to—and absorption by—a subject and can be included in the compositions of the present invention without causing a significant adverse toxicological effect on the patient. As used herein, these terms are intended to include any and all solvents, dispersion media, coatings, antibacterial and antifungal agents, antioxidant agents, isotonic and absorption delaying agents, and the like, compatible with pharmaceutical administration. Non-limiting examples of pharmaceutically-acceptable excipients include water, NaCl, normal saline solutions, lactated Ringer's, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients are useful in the present invention. The use of such media and agents for pharmaceutically active substances is well known in the art. Except insofar as any conventional media or agent is incompatible with the active compound, use thereof in the compositions is contemplated. Supplementary active compounds can also be incorporated into the compositions. One of ordinary skill in the art will recognize that other pharmaceutical excipients are useful in the present invention.

### E. Glucocorticoid Receptor Modulators (GRMs)

Exemplary GRMs comprising a heteroaryl ketone fused azadecalin structure include those disclosed and described in U.S. Pat. No. 8,859,774; in U.S. Pat. No. 9,273,047; in U.S. Pat. No. 9,707,223; and in U.S. Pat. No. 9,956,216, all of which patents are hereby incorporated by reference in their entireties. A GRM comprising a heteroaryl ketone fused azadecalin structure has the following structure:

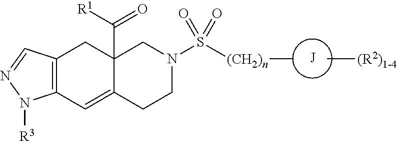

wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and —NR^{2a}R^{2b},
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

In embodiments, the GRM is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl) pyridin-2-yl)methanone, also known as “relacorilant”, which has the following structure:

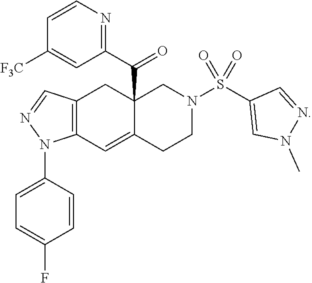 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

In embodiments, the GRM is the compound (R)-(1-(4-fluorophenyl)-6-((4-(trifluoromethyl)phenyl) sulfonyl)-4,4a, 5,6,7,8-hexahydro-1-H-pyrazolo P,4-g]isoquinolin-4a-yl)(pyridin-2-yl)methanone, also known as “dazucorilant”, which has the following structure:

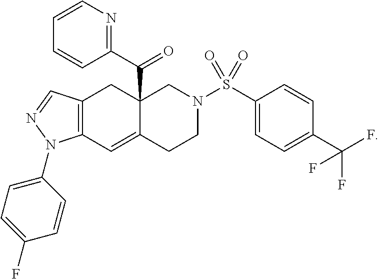 [SMILES: O=C(c1ccccn1)[C@]12Cc3cnn(-c4ccc(F)cc4)c3C=C1CCN(S(=O)(=O)c1ccc(C(F)(F)F)cc1)C2]

### PHARMACEUTICAL COMPOSITIONS AND ADMINISTRATION

In embodiments, the pharmaceutical compositions comprising a heteroaryl-ketone fused azadecalin GRM are administered to a patient suffering from an adrenal gland tumor, or is used for the treatment of an adrenal gland tumor.

A GRM comprising a heteroaryl-ketone fused azadecalin structure can be formulated in a suitable pharmaceutical formulation for the methods and uses disclosed herein. Such a pharmaceutical formulation may be prepared and administered in a wide variety of oral, parenteral and topical dosage forms. Oral preparations may include tablets, pills, powder, dragees, capsules, liquids, lozenges, gels, syrups, slurries, suspensions, etc., suitable for ingestion by the patient. A GRM comprising a heteroaryl-ketone fused azadecalin structure can also be administered by injection, that is, intravenously, intramuscularly, intracutaneously, subcutaneously, intraduodenally, intraperitoneally, intranasally, and by inhalation.

For preparing pharmaceutical compositions comprising a GRM comprising a heteroaryl-ketone fused azadecalin structure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

The pharmaceutical preparation is preferably in unit dosage form. In such form the preparation is subdivided into unit doses containing appropriate quantities of the active component, a GRM comprising a heteroaryl-ketone fused azadecalin structure. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of GRM comprising a heteroaryl-ketone fused azadecalin structure in a unit dose preparation may be varied or adjusted from 1 mg to 2000 mg, or 10 mg to 1000 mg, or 15 mg to 900 mg, or 20 mg to 800 mg, or 25 mg to 750 mg, or 50 mg to 500 mg. Suitable dosages also include about 1 mg, 5, 10, 15, 20, 25, 30, 40, 50, 60, 70, 75, 80, 90, 100, 150, 200, 250, 300, 400, 500, 600, 700, 750, 800, 900, or 1000 mg, according to the particular application and the potency of the active component. The composition can, if desired, also contain other compatible therapeutic agents.

GRMs comprising a heteroaryl-ketone fused azadecalin structure can be administered orally. For example, a GRM comprising a heteroaryl-ketone fused azadecalin structure can be administered as a pill, a capsule, or liquid formulation as described herein. In some embodiments, the GRM comprising a heteroaryl-ketone fused azadecalin structure is administered in one dose. In other embodiments, the GRM is administered in more than one dose, e.g., 2 doses, 3 doses, 4 doses, 5 doses, 6 doses, 7 doses, or more. The amount will vary according to, for example, the GRM properties and patient characteristics. Generally, administration of a GRM should be continued until clinically significant reduction or amelioration is observed; treatment may last for as long as two years or even longer, and may continue indefinitely as needed. In embodiments, GRMs comprising a heteroaryl-ketone fused azadecalin structure can be administered, e.g., intravenously (e.g., by injection or infusion) or via parenteral administration.

### EXAMPLES

The following examples are provided by way of illustration only and not by way of limitation. Those of skill will readily recognize a variety of noncritical parameters which could be changed or modified to yield essentially similar results.

### Example 1

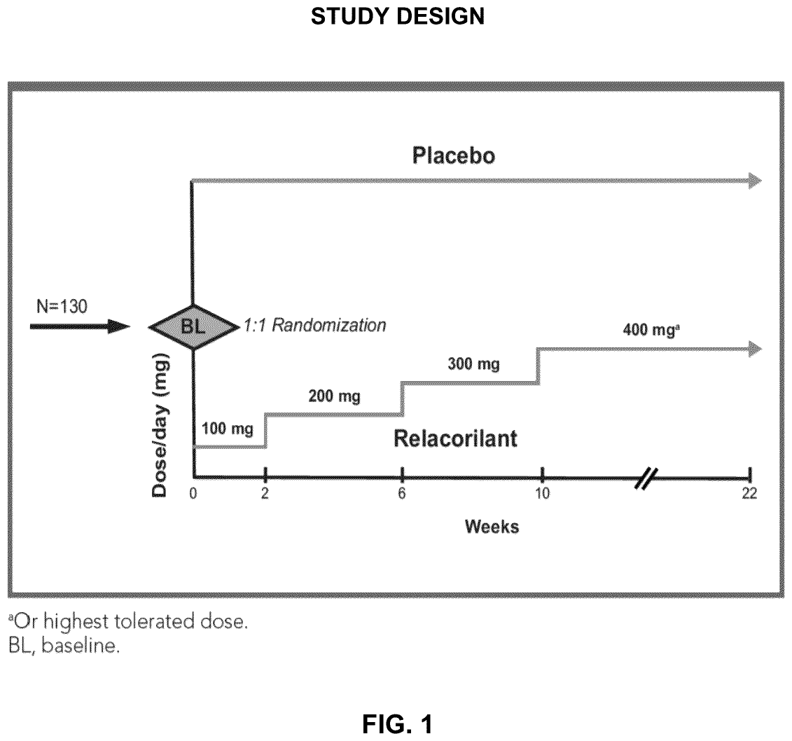

The following protocol describes the GRADIENT clinical trial, an on-going, randomized, double-blind, placebo-controlled Phase 3 study which focuses on patients with hypercortisolism secondary to adrenal adenoma(s) or hyperplasia (ClinicalTrials.gov Identifier: NCT04308590). This study includes patients with cortisol-secreting adrenal adenomas or hyperplasia associated with DM/IGT (diabetes mellitis (DM) and/or impaired glucose tolerance (IGT)) and/or uncontrolled systolic hypertension. The study enrolled patients with hypercortisolism due to cortisol secreting adrenal adenomas or hyperplasia. These patients may or may not present the classic clinical manifestations of overt Cushing syndrome (i.e., facial appearance, buffalo hump, striae, etc.). Patients were identified as suffering from cortisol-secreting adrenal gland tumors based on abnormal dexamethasone suppression test (DST) and low ACTH. Patients could receive medications that were commonly used to treat diabetes, hypertension, or dyslipidemia if required. Following screening, those patients meeting study criteria were randomly assigned to receive doses of either placebo or relacorilant for 22 weeks of treatment. Patient follow-up was within 28 days of the last dose. Total patient participation in the study was up to 32 weeks. The GRADIENT study design is illustrated in FIG. 1. Patients discussed herein were participants in the GRADIENT study.

As indicated in FIG. 1, the dose of relacorilant was increased sequentially in a step-wise manner from 100 mg orally once daily to a target dose of 400 mg once daily based on tolerability and improvement in hyperglycemia and/or hypertension. The starting dose at Baseline was 100 mg relacorilant or matching placebo once daily for 2 weeks, after which the dose was increased to 200 mg. As tolerated, the dose was then increased by 100 mg to 300 mg once daily after 4 weeks, and, as tolerated, the dose was then increased to 400 mg once daily after another 4 weeks. If a dose was not tolerated, dose interruption, dose reduction, and dose re-escalation were allowed. Patients were maintained at a once-daily dose of 400 mg, or at their highest tolerated dose. Dose titrations after 18 weeks were not permitted. Patients who completed the study were permitted to continue relacorilant treatment in an extension study if they have at least 80% adherence with scheduled dosing (by capsule counts), and are expected to maintain clinical benefit from the study drug. Although not expected to regress, adrenal tumors progress very slowly, and rarely progress into overt Cushing syndrome (Fassnacht et al. 2016 Eur J Endocrinol. 175 (2):G1-G34. doi: 10.1530/EJE-16-0467). Patients receiving placebo continued to receive their previously prescribed medications; thus, although the placebo was not expected to provide additional benefit to those patients receiving placebo, neither was any detriment expected.

Relacorilant was supplied as 100 mg capsules for oral dosing. Patients in the placebo control arm received placebo matched to study drug, supplied as 100 mg capsules for oral dosing. Oral dosing was administered without food to fasted patients before breakfast. Although relacorilant effects were expected to be observable after about 3 months of treatment, patients were treated with relacorilant for at least 6 months prior to adrenal imaging. The results presented herein were obtained with computed tomography (CT) scans; however, any method of visualizing the adrenal glands and adrenal tumors may be used (e.g., magnetic resonance imaging (MRI), ultrasound imaging, or any other method effective to identify and/or visualize a subject's adrenal glands and adrenal tumors (if any)).

This study was designed to enroll approximately 130 patients (ages 18-80 years) to be assigned to either the impaired glucose tolerance/diabetes mellitus (IGT/DM) or hypertension (HTN) subgroup. Key inclusion criteria included:
Patient has an adrenal adenoma or adrenal hyperplasia;
Patient lacks cortisol suppression (>1.8 mcg/dL serum cortisol) on either a 1 mg overnight dexamethasone suppression test (DST) or on a 2-mg 48-hour DST (Fassnacht M et al. Management of adrenal incidentalomas—a European Society of Endocrinology Clinical Practice Guideline in collaboration with the European Network for the Study of Adrenal Tumors. 2023.)
Patient exhibits suppressed or low (≤15 picograms per milliliter (pg/mL)) early-morning ACTH levels on at least 2 occasions.

Inclusion criteria further include either Glucose Impairment or Uncontrolled Systolic Hypertension, where these criteria include at least one of the following:

### Glucose Impairment:

Impaired Glucose Tolerance (IGT): Plasma glucose ≥140 mg/dL and <200 mg/dL on 2-hour OGTT; or
Diabetes Mellitus (DM): Fasting plasma glucose ≥126 mg/dL, and/or plasma glucose and <200 mg/dL on 2-hour oral glucose tolerance test (OGTT), or Hb1Ac≥6.5%.

### Uncontrolled Systolic Hypertension:

Mean systolic blood pressure (SBP)≥130 to ≤170 mm Hg based on 24-hour ambulatory blood pressure monitoring (ABPM).

Key exclusion criteria, for which potential patients are excluded from the study, include:
severe, uncontrolled hypertension (mean SBP>170 mmHg or mean diastolic blood pressure (DBP)>110 mmHg; based on 24-hour ABPM);
poorly controlled diabetes mellitis (HbA1c>12%);
Adrenocortical carcinoma;
Autonomous co-secretion of aldosterone;
Uncontrolled, clinically significant hypo- or hyperthyroidism.

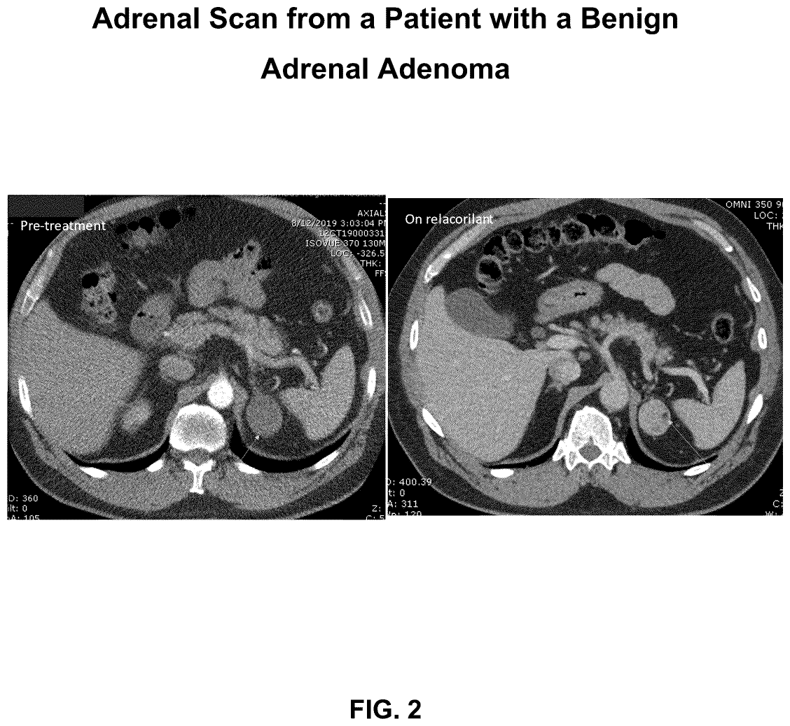

FIG. 2 presents adrenal CT scans obtained from a patient with a benign adrenal adenoma. The tumor was determined to be a cortisol-secreting tumor based on the patient's abnormal DST and low ACTH levels.

The two CT scans presented in FIG. 2 were taken from this patient at two different times: the first scan (on the left) was taken in 2019, before relacorilant treatment, and the second scan (on the right) was taken in 2021 after the patient had been receiving daily doses of relacorilant, beginning at 100 mg/day and increased in 100 mg/day steps to 400 mg/days. The size of this adrenal gland tumor was reduced following relacorilant treatment. The patient had received daily relacorilant administration for almost two years prior to the time at which the image on the right was obtained. By that time, the adenoma had decreased in size as compared to the initial size (initial size of 4.2 cm reduced to 3.1 cm with relacorilant treatment), and also, after relacorilant treatment, exhibited areas of degeneration (little black areas inside the adenoma) that were not present in the initial scan. Arrows point to the adrenal tumor in both images. The arrow in the right image it also points a degenerative area inside the tumor that suggests an apoptotic process. The plasma ACTH of this patient was undetectable prior to relacorilant administration. ACTH levels normalized at 6 months of relacorilant administration, and has remained within the normal range since then (between 15-18 pg/ml).

The levels of ACTH in the blood plasma of this patient were undetectable prior to relacorilant treatment. Since pituitary ACTH secretion is regulated by feedback sensitive to the levels of cortisol in the blood, a cortisol-secreting adrenal tumor would be expected to lead to low or absent ACTH levels via this feedback mechanism. After 6 months of relacorilant treatment, the ACTH levels of this patient normalized, and have been between about 15 picograms per milliliter (pg/mL) to about 18 pg/mL since then (these ACTH levels are within the normal range). Cortisol levels decreased and the DST normalized suggesting resolution of the cortisol excess.

The natural history of adrenal adenomas it to grow over time; such adenomas do not typically regress. Mifepristone treatment of Cushing's syndrome patients leads to increased ACTH and increased cortisol levels, which increases can themselves increase the size of an adenoma, so that growth of the tumor, not shrinkage, would be expected with mifepristone treatment.

Surprisingly, the adrenal scans shown in FIG. 2 show that a) the size of the adrenal tumor decreased following treatment with relacorilant, and b) that the tumor developed fat droplets (see arrows pointing to some fat droplets) indicating degeneration within the tumor. Such unexpected tumor shrinkage should lead to decreased cortisol secretion from the tumor. In such case, treatment of the patient could be adjusted, where the dose of relacorilant to be administered should then be decreased in accordance with the decrease in cortisol secretion.

### Example 3. Further Example

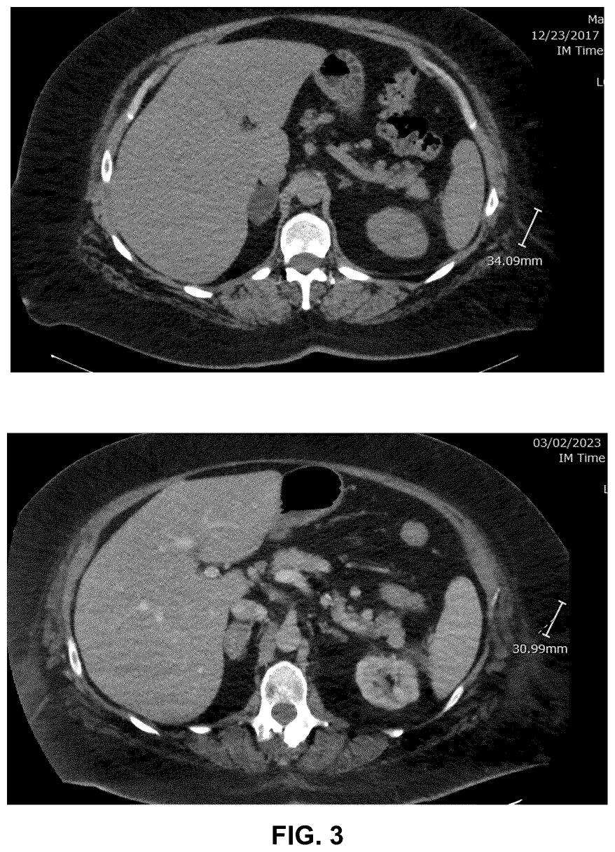

This Example presents a further patient experiencing shrinkage of a cortisol secreting adrenal adenoma after receiving relacorilant treatment as shown in the abdominal computed tomography (CT) scan images of FIG. 3. As noted above, adrenal adenoma tumors typically increase in size over time. In contrast with such expected increase, the tumor in this patient was smaller following relacorilant treatment than it had been prior to relacorilant treatment. The patient's initial CT scan, taken prior to relacorilant treatment, showed an adrenal nodule (tumor) that was measured to be 3.5×2.5 cm in size (image taken in December 2017). The measurement line in the upper image of FIG. 3 (shown at the right of the image) indicates 34.08 millimeters (mm). The lower image was taken in 2023, after the patient had received at least 3 months of daily relacorilant, beginning at a daily dose of 100 mg/day which was adjusted in 100 mg/day increments during treatment to doses of 200 mg/day, 300 mg/day, and up to a maximal dose of 400 mg/day. The patient's latest CT scan (image taken in March 2023) shows the nodule to be no larger than 3.1×2.3 cm. The measurement line in the lower images indicates a reduced dimension of 30.99 mm. The size of this adrenal gland tumor was reduced following relacorilant treatment.

All patents, patent publications, publications, and patent applications cited in this specification are hereby incorporated by reference herein in their entireties as if each individual publication or patent application were specifically and individually indicated to be incorporated by reference. In addition, although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of reducing the size of an adrenal gland tumor in a patient suffering therefrom,
wherein said reducing the size of said adrenal gland tumor comprises one or more of the group consisting of reducing the size of, reducing a dimension of, reducing the volume of, reducing the weight of, reducing the mass of, causing degenerative changes in, inducing apoptosis in at least part of, preventing the growth of, slowing the growth of, reducing secretion from, and altering the composition of hormones secreted from, an adrenal gland tumor in said patient,
the method comprising administering an effective amount of a glucocorticoid receptor modulator (GRM) to the patient, wherein the GRM comprises a heteroaryl-ketone fused azadecalin structure of formula (I), 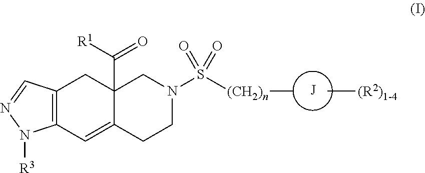
wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and —NR^{2a}R^{2b},
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl;
subscript n is an integer from 0 to 3;
or salts and isomers thereof,
effective to reduce the size of said adrenal gland tumor.

2. The method of claim 1, wherein said GRM is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone (relacorilant), which has the structure:

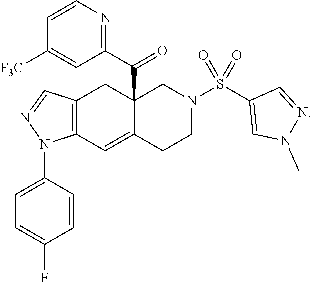 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

3. The method of claim 1, wherein the GRM is administered orally.

4. The method of claim 1, wherein the GRM is administered with food.

5. The method of claim 1, wherein the adrenal gland tumor is a cortisol-secreting adrenal gland tumor.

6. The method of claim 5, wherein the administration of an effective amount of GRM comprising a heteroaryl-ketone fused azadecalin structure is effective to reduce the secretion of cortisol by said adrenal gland tumor

7. The method of claim 1, wherein the patient suffers from abnormally high blood levels of cortisol, and the treatment is effective to reduce cortisol blood levels in the patient.

8. The method of claim 1, wherein the patient lacks an appropriate diurnal rhythm in their cortisol levels.

9. The method of claim 1, wherein the patient suffers from cortisol excess, exhibits symptoms of Cushing's syndrome, or both, and wherein the administration of an effective amount of GRM comprising a heteroaryl-ketone fused azadecalin structure is effective to treat and reduce the severity of said cortisol excess, said symptoms of Cushing's syndrome, or both cortisol excess and Cushing's syndrome symptoms, in the patient.

10. The method of claim 1, wherein the patient exhibits symptoms of Cushing's syndrome selected from one or more of hypercortisolism, hypertension, hypercoagulopathy, hyperglycemia, glucose intolerance, obesity, dyslipidemia, muscle weakness, cognitive dysfunction, osteoporosis (e.g., low bone density with increased risk of fractures), skin lesions, upper body fat, moon face, depression, and a suppressed immune system, wherein the administration of an effective amount of GRM comprising a heteroaryl-ketone fused azadecalin structure is effective to treat and reduce the severity of said symptoms of Cushing's syndrome in the patient.

11. A method of reducing the tumor load of an adrenal gland tumor in a patient suffering from said adrenal gland tumor, wherein said reducing the tumor load comprises one or more of reducing the size of, reducing a dimension of, reducing the volume of, reducing the mass of, reducing the weight of, causing degenerative changes in, inducing apoptosis in at least part of, preventing the growth of, slowing the growth of, reducing secretion from, and altering the composition of hormones secreted from one or more adrenal gland tumor(s) in said patient,
the method comprising administering an effective amount of a glucocorticoid receptor modulator (GRM) to the patient, wherein the GRM comprises a heteroaryl-ketone fused azadecalin structure of formula (I), 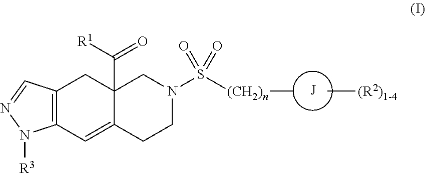
wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and —NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl;
subscript n is an integer from 0 to 3;
or salts and isomers thereof,
effective to reduce the tumor load of adrenal gland tumor(s) in a patient suffering therefrom.

12. The method of claim 11, wherein said GRM is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone (relacorilant), which has the structure:

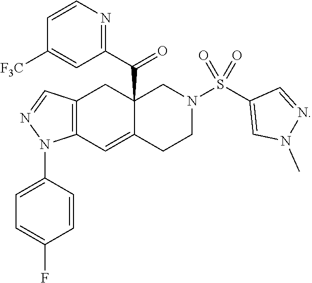 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

13. The method of claim 11, wherein the GRM is administered orally.

14. The method of claim 11, wherein the GRM is administered with food.

15. The method of claim 11, wherein the adrenal gland tumor is a cortisol-secreting adrenal gland tumor.

16. The method of claim 15, wherein the administration of an effective amount of GRM comprising a heteroaryl-ketone fused azadecalin structure is effective to reduce the secretion of cortisol by said adrenal gland tumor.

17. The method of claim 11, wherein the patient suffers from abnormally high blood levels of cortisol, and said treatment is effective to reduce cortisol blood levels in the patient.

18. The method of claim 11, wherein the patient lacks an appropriate diurnal rhythm in their cortisol levels.

19. The method of claim 11, wherein the patient suffers from cortisol excess, exhibits symptoms of Cushing's syndrome, or both, and wherein the administration of an effective amount of GRM comprising a heteroaryl-ketone fused azadecalin structure is effective to treat and reduce the severity of said cortisol excess, said symptoms of Cushing's syndrome, or both cortisol excess and Cushing's syndrome symptoms, in the patient.

20. The method of claim 11, wherein the patient exhibits symptoms of Cushing's syndrome selected from one or more of hypercortisolism, hypertension, hypercoagulopathy, hyperglycemia, glucose intolerance, obesity, dyslipidemia, muscle weakness, cognitive dysfunction, osteoporosis (e.g., low bone density with increased risk of fractures), skin lesions, upper body fat, moon face, depression, and a suppressed immune system, wherein the administration of an effective amount of GRM comprising a heteroaryl-ketone fused azadecalin structure is effective to treat and reduce the severity of said symptoms of Cushing's syndrome in the patient.

21. The method of claim 1, wherein the tumor is a benign adrenal gland tumor.

22. The method of claim 11, wherein the tumor is a benign adrenal gland tumor.
